(19) United States
(12) Patent Application Publication
(10) Pub. No.: US 2022/0122727 A1
(43) Pub. Date: Apr. 21, 2022

(54) APPARATUS AND METHOD FOR GENERATING NARRATIVE FOR LIFESTYLE RECOGNITION

(71) Applicant: Electronics and Telecommunications Research Institute, Daejeon (KR)

(72) Inventors: Jiyoun LIM, Daejeon (KR); Hyuntae JEONG, Daejeon (KR); Ga Gue KIM, Daejeon (KR); Kyoung Ju NOH, Daejeon (KR); Jeong Mook LIM, Daejeon (KR); Seungeun CHUNG, Daejeon (KR); Chiyoon JEONG, Daejeon (KR)

(73) Assignee: Electronics and Telecommunications Research Institute, Daejeon (KR)

(21) Appl. No.: 17/503,051

(22) Filed: Oct. 15, 2021

(30) Foreign Application Priority Data
Oct. 16, 2020 (KR) 10-2020-0134238

(51) Int. Cl. G16H 40/67; G16H 50/70; G16H 50/30; G16H 20/70

(52) U.S. Cl. CPC G16H 40/67; G16H 20/70; G16H 50/30; G16H 50/70

(57) ABSTRACT

Provided are an apparatus and method for generating a narrative for lifestyle recognition. The apparatus for generating a narrative for lifestyle recognition includes an input unit configured to collect basic data of narrative generation for the lifestyle recognition, a memory configured to store a program for generating a user's lifestyle narrative using the basic data, and a processor configured to execute the program, in which the processor performs a causal relationship analysis on a cause of a mind state and a result of the mind state using the lifestyle narrative.

### CROSS-REFERENCE TO RELATED APPLICATION

This application claims priority to and the benefit of Korean Patent Application No. 10-2020-0134238, filed on Oct. 16, 2020, the disclosure of which is incorporated herein by reference in its entirety.

### BACKGROUND

### 1. Field of the Invention

The present invention relates to an apparatus and method for generating a narrative for lifestyle recognition.

### 2. Discussion of Related Art

According to the related art, lifestyle-related data and information are obtained through questionnaires and consultations, which cause a problem in that information loss occurs in a way that depends on a subject's memory.

In addition, according to the related art, there is a limitation in not being able to provide services for automatically recognizing personal narratives using objective data, mental health-related services using lifestyle information, and the like.

In addition, according to the related art, there is a limitation in not being able to clearly reveal the causal relationship between complexity of a person's lifestyle and events by focusing on recognizing events that repeatedly occur at the same time.

### SUMMARY OF THE INVENTION

The present invention is directed to providing an apparatus and method for generating a narrative that are capable of collecting objective data, recognizing a user's context, and extracting a narrative of a user's personal lifestyle.

According to an aspect of the present invention, there is provided a system for generating a narrative for lifestyle recognition including: a server configured to receive and store multi-modal sensor data and user's device usage log data; a computing device configured to call the multi-modal sensor data and the device usage log data from the server to recognize contextual information, generate a user's personal narrative, and derive lifestyle information; and a display device configured to perform visualization using the lifestyle information and provide an option to confirm an analysis result.

The multi-modal sensor data may include biometric information and environmental information.

The computing device may preprocess the multi-modal sensor data and the device usage log data to be synchronized and integrated based on time.

The computing device may recognize the contextual information, which includes a user's behavior, social life, time and space, and mind state, by time.

The computing device may classify the narrative based on a predefined time and extract patterns for a habit narrative and a non-habit narrative.

The computing device may derive a habit index using the patterns for the habit narrative and the non-habit narrative and derive a lifestyle pattern, which affects the mind state, using the habit index.

The display device may provide a responsive user interface to which at least one option of an inquiry period, a time interval, confirmation of analysis results for each mind state, and confirmation of analysis results by location is applied.

According to another aspect of the present invention, there is provided a method of generating a narrative for lifestyle recognition including: (a) collecting and preprocessing user's biometric information, activity information, and environmental information; (b) forming an episode including of contextual information including a user's behavior, social life, time and space, and mind state using the data preprocessed in operation (a); (c) grouping the episode to generate sequence data and interpreting the generated data as a user's lifestyle narrative; (d) extracting a habit pattern and a non-habit pattern using the narrative; and (e) analyzing a correlation between the user's lifestyle and mind state.

In operation (a), information including multi-modal sensor data and device usage log data may be collected and preprocessed for synchronization based on time.

In operation (b), the episode may be generated for each time period according to an order of generation and duration information.

In operation (c), the sequence data may be generated by dividing the episode into groups of a preset time unit.

In operation (d), sequential pattern mining may be applied to extract the habit pattern for a collected period, and a user's lifestyle narrative may be generated as a pattern including consecutive events.

In operation (d), a habit index may be extracted for each category of the contextual information.

According to still another aspect of the present invention, there is provided an apparatus for generating a narrative for lifestyle recognition including: an input unit configured to collect basic data of narrative generation for the lifestyle recognition; a memory configured to store a program for generating a user's lifestyle narrative using the basic data; and a processor configured to execute the program, in which the processor performs a causal relationship analysis on a cause of a mind state and a result of the mind state using the lifestyle narrative.

The input unit may collect data including biometric information, environmental information, and activity information.

The processor may generate contextual information including a user's behavior, social life, time and space, and mind state, and extract a pattern for a habit or non-habit narrative.

The processor may extract a habit pattern by applying sequential pattern mining, and generate the user's lifestyle narrative using a pattern including consecutive events.

The processor may derive a habit index using the pattern, and derive a lifestyle pattern, which affects a specific mind state, using the habit index.

The processor may calculate the habit indexes for each of behavior, social relationship, time and space, and emotion.

The above-described configurations and operations of the present invention will become more apparent from embodiments described in detail below with reference to the drawings.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 illustrates a system for generating a narrative for lifestyle recognition according to an embodiment of the present invention;

FIG. 2 illustrates an analysis module according to an embodiment of the present invention;

FIG. 3 illustrates an analysis process of the recognition module according to the embodiment of the present invention;

FIG. 4 illustrates a lifestyle and a causal relationship analysis process of the analysis module according to the embodiment of the present invention;

FIG. 5 illustrates an example of a lifestyle analysis result according to the embodiment of the present invention; and

FIG. 6 illustrates an apparatus for generating a narrative for lifestyle recognition according to an embodiment of the present invention.

### DETAILED DESCRIPTION OF EXEMPLARY EMBODIMENTS

The above-described objects and other objects of the present invention and methods of accomplishing them will become apparent from the following description of embodiments with reference to the accompanying drawings.

However, the present invention is not limited to the embodiments disclosed below but may be implemented in various different forms. Only the following embodiments are provided to easily inform those of ordinary skill in the art to which the present invention pertains of the object, configuration and effect of the invention, and the scope of the present invention is defined by the description of the claim.

Meanwhile, terms used in the present specification are for describing the embodiments rather than limiting the present invention. Unless otherwise stated, a singular form includes a plural form in the present specification. Components, steps, operations, and/or elements being described by the terms “comprise” and/or “comprising” used in the present invention do not exclude the existence or addition of one or more other components, steps, operations, and/or elements.

Hereinafter, in order to help those skilled in the art to understand, the present invention will first describe the proposed background and then describe embodiments of the present invention.

A person's lifestyle is not defined by how often a single activity occurs but may be described as a narrative in which duration of each activity and an order of generation of complex activities are complexly connected.

According to the related art, recognition of narratives on lifestyle was not carried out through a technical approach but through methods such as questionnaires or interviews that depend on individual's memory in the field of social science or psychology.

However, these methods have the potential to cause loss of important information due to the limitation of human memory.

People make efforts to recognize and improve lifestyle habits for health care.

According to the related art, a service is provided to record a life log using a smartphone app to identify a state of a person by himself or herself.

In addition, a service that collects and analyzes data using wearable devices such as a smart band and recognizes, visualizes, and provides an activity amount, exercise information, a sleep state, etc., for a certain period without user intervention is provided.

According to the related art, a technology for identifying a user's lifestyle has been proposed, and these methods define, as a daily lifestyle, activities that repeatedly appear at a specific time as a habit, or activities that are not specific situations such as risk situations for specific targets, such as the elderly and patients with high-risk diseases. In addition, the method of automatically extracting user's activity, sleep, and mind state patterns using wearable sensor data has been proposed.

However, according to the related art, there is a limitation in not proposing a technology for automatically recognizing an individual's narrative using objective data and not providing a service using lifestyle information related to mental health such as mental state or stress.

An individual's narrative includes duration of an activity and an order of generation of complex activities and is an important concept forming a person's lifestyle.

In the field of psychiatry and psychology, lifestyle-related data and information are obtained through questionnaires and counseling and are used for diagnosis and treatment.

In the treatment of depression, stress, sleep disorders, or the like, patients are asked questions about a behavior and habit related to a mind state, surrounding environmental information, etc. In this case, information that depends on a subject's memory about what happened for as short as a week or as long as a month is acquired.

However, according to a forgetting curve of Hermann Ebbinghaus, humans only remember 33% of learned facts after a day, 28% after two days, and 21% after a month. Therefore, information loss inevitably occurs in questionnaires about the state of the past week or month.

In addition, according to the related art, there is a limitation in not being able to propose a technology for automatically acquiring lifestyle information by forming a narrative for the lifestyle information that is the cause of the mind state based on a causal relationship.

In order to solve the above-described problems, the present invention proposes an apparatus and method for generating a narrative for extracting a narrative of a user's personal lifestyle by collecting objective data.

According to an embodiment of the present invention, by using a multi-modal sensor capable of collecting data on a user's behavior, bio-signals, and environment and heterogeneous data acquired from a mobile device, an individual's lifestyle according to an emotional health state such as a psychological state and stress is generated in a narrative form.

The user device collects user-related data and transmits the collected data to a server through a network service so that the transmitted collected data is stored in the server.

The computing device calls the user's multi-modal sensor and device data from the server and synchronizes and integrates each piece of information based on time to preprocess the synchronized and integrated information.

The computing device analyzes the preprocessed data to recognize user's contextual information by time.

The contextual information includes user's behavior, social life, time and space, and mind state (behavior, social, geospatial, and mind-state) by time.

The contextual information recognized by the computing device is used for analysis that forms the user's personal narrative using the precedent and precedent relationship and the duration of the behavior.

An analysis module of the computing device generates sequence data based on a predefined time to classify an individual's narrative and performs pattern extraction analysis representing a habit/non-habit narrative.

The computing device derives a habit index representing a degree of conformity to an individual's lifestyle for each day by using a habit/non-habit pattern.

The habitual/non-habit pattern and the habit index can be derived for each piece of contextual information, that is, each of behavior, social life, time and space, and mind state.

In the analysis module, a causal relationship analysis is performed using a behavior representing a lifestyle, a habit index for social life and time and space, a mind habit index representing the mind state, stress characteristics, and a sleep state, and derives a lifestyle pattern that has a decisive influence on a specific mind state using the analysis performance results.

The concept of “narrative” according to the embodiment of the present invention is differentiated from the concept in which the habit or lifestyle is repeated at the same time.

A display device (service device) visualizes the analyzed information so that a user himself/herself, a guardian, or a medical professional who manages a user's mind state can confirm the analysis result.

The display device (service device) provides a responsive user interface to which various options, such as an inquiry period for a user to inquire about necessary information, a time interval expressed in visualization, time such as weekdays/weekends, confirmation of the analysis results for each mind state, and confirmation of the analysis results by location using user's location information, are applied.

According to an embodiment of the present invention, a user's mental health management is supported by providing objective lifestyle information including narratives to a user himself/herself, a guardian, or a medical professional.

According to an embodiment of the present invention, when users have a questionnaire or consultation in a situation where the users get help from medical professionals, it is possible to use objective lifestyle information including narratives, and through this, the medical professionals make medical decisions that can actually help improve patients' mental health based on users' personal characteristics and data-based objectivity information.

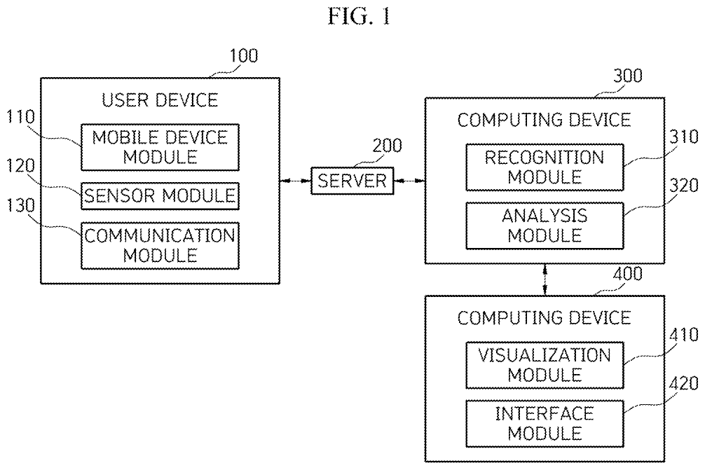

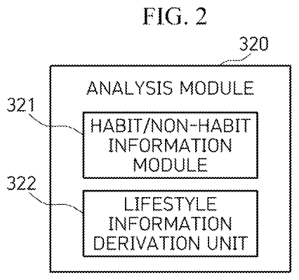

FIG. 1 illustrates a system for generating a narrative for lifestyle recognition according to an embodiment of the present invention, and FIG. 2 illustrates an analysis module according to an embodiment of the present invention.

The system for generating a narrative for lifestyle recognition according to the embodiment of the present invention recognizes complex activities using multi-modal sensor data and user's device usage log data and extracts sequence patterns in consideration of the duration and the order of generation.

The user device 100 includes a mobile device module 110, a sensor module 120, and a communication module 130.

The mobile device module 110 collects a user's device usage log indicating information on a social activity context, such as a phone call using a smartphone, and social app usage information.

A multi-modal sensor of the sensor module 120 includes an acceleration sensor, a gyro sensor, a geomagnetic sensor, a skin conductivity sensor, a skin temperature sensor, a heart rate sensor, an electrocardiogram sensor, a global positioning system (GPS), etc., and collects direct signals about users, such as body movement signals and bio signals, and environmental signals such as a location for a GPS and Wi-Fi, illuminance, and sound.

The communication module 130 transmits the collected signal to a server 200 through a network service, and the server 200 stores the collected signal.

The computing device 300 includes a recognition module 310 and an analysis module 320.

The recognition module 310 calls and preprocesses data stored in the server 200 and recognizes behavior, social life, time and space, and a mind state (behavior, social, geospatial, and mind-state) as contextual information.

In this case, the recognition module 310 recognizes the mind state by recognizing stress, an emotional state, a sleep state, and the like.

The analysis module 320 includes a habit/non-habit information extraction unit 321 and a lifestyle information derivation unit 322.

The habit/non-habit information extraction unit 321 receives the user contextual information from the recognition module 310 to generate a user's individual narrative, and extracts the habit/non-habit information using the generated user's individual narrative.

The lifestyle information derivation unit 322 analyzes the causal relationship between the user's lifestyle including the habit/non-habit information and the mind state to derive the user's lifestyle information which is a cause of a specific mind state.

According to the embodiment of the present invention, the analysis result derived from the computing device 300 is visualized on a display device 400 so as to be easily understood by a user and includes a responsive user interface that provides an option to confirm detailed information of a necessary condition.

That is, the display device 400 includes a visualization module 410 that visualizes the results of the analysis module and an interface module 420 that provides an option to be able to confirm the analysis results according to time, location, emotion, etc. and provides a responsive user interface.

By the visualization module 410, the narrative is expressed in a way that may express a story, such as a sequence pattern form, a sentence, and the like.

The interface module 420 provides the responsive user interface to which options, such as an inquiry period for a user to inquire about necessary information, a time interval expressed in the visualization, time such as weekdays/weekends, confirmation of the analysis results for each mind state, and confirmation of the analysis results by location using user's location information, are applied.

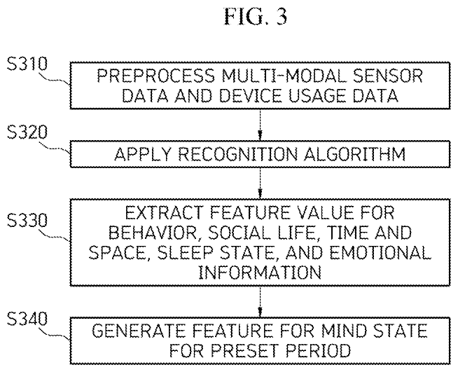

FIG. 3 illustrates an analysis process of the recognition module 310 according to the embodiment of the present invention.

The recognition module 310 calls multi-modal sensor data and device usage data from the server 200, preprocesses a missing value, a logical error, etc., and integrates data through time synchronization of heterogeneous sensor data (S310).

The recognition module 310 applies a recognition algorithm including machine learning techniques such as deep learning (S320) and recognizes feature values for the user's behavior, social life, time and space, sleep state, and emotional information (S330).

A user's behavior includes waking up, moving, work, eating, exercising, etc. which may happen for a user's preset period (for example, a day).

In this case, the classification of behavior may follow a classification system of a life time survey defined according to lifestyles and cultures by country in each national statistical office, and users may also define and use the required behavior classification.

In the case of performing an activity with others, social life information is information including a category of people who do behavior together, the number of people who do behavior together, etc.

The time and space information is information on a time and location where the user's behavior happens using time values of a sensor and a device and GPS information.

The sleep state information is information on the sleep state acquired using the user's movement and bio-signals.

The emotional information is recognized on a 5-point or 7-point scale of arousal and valence using sensor data as an input value.

The recognition module 310 processes the sleep state and emotional information to generate features of the mind state by preset period (for example, for each day, daily) (S340). As such, the mind state is described by being divided into stress, an emotional state, and a sleep state.

The analysis module 320 according to the embodiment of the present invention analyzes a lifestyle using the result derived from the recognition module 310 and performs the causal relationship analysis between the lifestyle and the mind state.

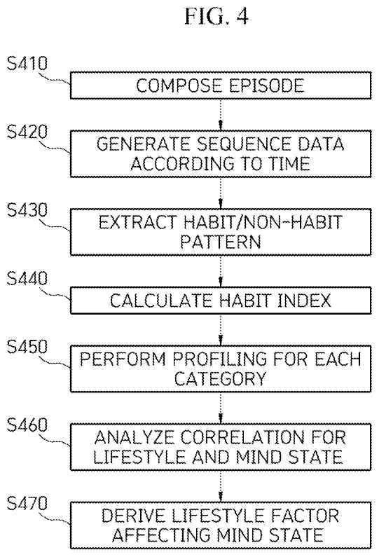

FIG. 4 illustrates a lifestyle and a causal relationship analysis process of the analysis module 320 according to the embodiment of the present invention.

In operation S410, an episode is composed in consideration of the order of generation and duration by using the user's behavior, social life, time and space, and emotional information.

The composed episode is expressed in the form of “movement (30 minutes)→work (120 minutes)→learning (30 minutes) . . . .”

In operation S420, sequence data for each time is defined and generated using the generated episode.

To compose the sequence data, a time (time-slot) may be divided into a time of morning (9:00 to 12:00), lunch (12:00 to 14:00), afternoon (14:00 to 18:00), evening (18:00 to 21:00), night (21:00 to 24:00), dawn (00:00 to 06:00), and early morning (06:00-09:00), which are usually used, and may be arbitrarily defined according to the user's needs.

By combining the social life, the time and space, and the emotional information based on behaviors within time, and generating the sequence data according to the order of generation and the duration information, it is possible to compose episodes by time.

When the episodes by time are divided into groups on a daily basis in operation S420, sequence data for representing daily episodic sequential patterns can be generated, and such sequence data is interpreted as a user's lifestyle narrative.

In operation S430, a habit pattern for the collected period is extracted by applying sequential pattern mining to the user's sequence data representing the lifestyle narrative as an input value.

The habit pattern extraction according to the embodiment of the present invention represents the narrative of the user's lifestyle as a pattern including a series of events, not about how often a specific event repeats at a specific time.

For example, describing user A's data as an example, if it is assumed that a pattern “travel by car→work with colleagues at work→study with colleagues→travel→social activities with friends→eat with friends” and a pattern “exercise with family→social activity with family” are a habit pattern of user A, and a pattern “leisure time outside alone→social activities with friends” is a rare pattern, according to the pattern information that forms the user A's lifestyle in a narrative form, and thus it can be confirmed that the user A seldom engages in leisure activities alone and meet his/her friends.

In operation S440, a habit index is calculated by a preset period (for example, daily) using the habit/non-habit pattern extracted in operation S430.

The daily habit index is an index indicating how frequently or continuously over a long period of time a user repeats patterns for a day in a data collection period.

The daily habit index may be calculated for each of the behavior, social relationship, time and space, and emotion.

In operation S450, for each day it is confirmed how similarly a day is spent compared to usual days through a behavior habit index, a social relationship habit index, a time and space habit index, and an emotional habit index.

That is, when the behavior habit index is high, it may be determined that a user may spend a day that may be defined as the user's lifestyle based on the behavior.

The user A of the above-described example may spend the day according to a usual lifestyle of waking up in the morning, preparing breakfast, moving to an office to work, studying, and meeting friends to eat.

On the other hand, when the time and space habit index is low, it is determined that the day is a different day from the user's usual day, that is, a day deviating from the lifestyle based on the time and space.

An example thereof may be a day in which a user spends a day which includes an unusual pattern such as a pattern in which the user is near his/her home and work in the morning but moves to a remote area that is not normally visited in the afternoon, stays for a few hours, and then moves to another remote area.

Habitual days and non-habitual days may be distinguished based on the daily habit index, and, if necessary, stages from habit to non-habit may be divided into more subdivided groups.

In operation S450, through this method, patterns of sequence data forming each group and information for identifying a lifestyle from the sequence data are profiled.

In operation S460, the correlation between the lifestyle expressed by the daily sequence pattern and the habit index and the daily mind state is analyzed using the lifestyle analysis result.

In this case, as input values, values for the stress, the emotional state, and the sleep state indicating the habit index and the mind state for the behavior, the social relationship, the time and space, and the emotion indicating the lifestyle derived from daily sequence patterns are input.

In operation S460, in order to recognize the daily sequence pattern as the lifestyle, a unique cycle for each lifestyle according to short-term/long-term effects is considered.

For example, a short cycle is based on a daily lifestyle, a cycle with a medium length is based on a weekday/weekend cycle, and when a long-term lifestyle analysis is required, it is based on a long cycle such as a quarter.

In order to consider the unique cycle, a causal relationship or a prediction algorithm considering seasonal factors is applied.

In this case, as an example of an applicable algorithm, moving average-based forecasting analysis may be applied to data with a short period, and seasonal forecasting analysis may be applied to data with a long period, and furthermore, analysis methods such as Hidden Markov chain and path analysis may be applied.

In operation S470, a lifestyle factor that affects a specific mind state of a user is derived by using the correlation analysis result.

For example, when the habitual behaviors and the time and space patterns on weekdays continue, stressful and negative mind states may persist.

In addition, when the social activity and the time and space patterns on the weekend are different from the usual lifestyle, the mind pattern of the following week may continue to maintain a positive state.

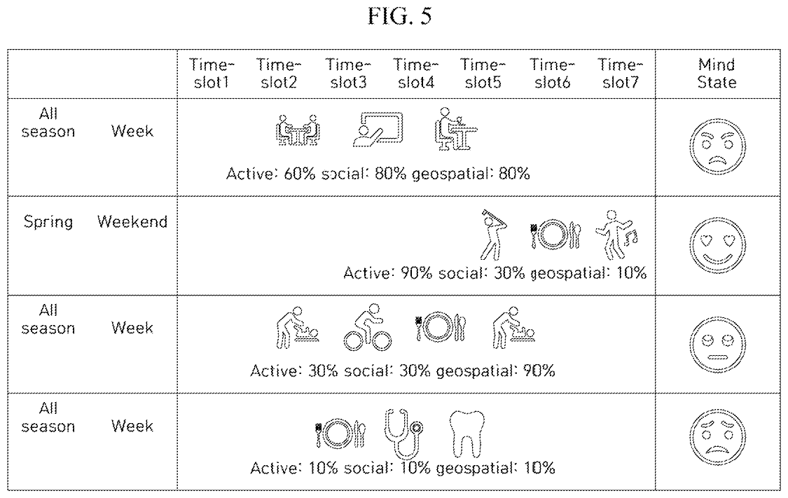

FIG. 5 illustrates an example of the lifestyle analysis result according to the embodiment of the present invention.

The interpretation according to the causal relationship analysis result is expressed as a behavior or a context sequence pattern as illustrated in FIG. 5. As a result, it can be confirmed that the user lifestyle narrative has a significant effect on a specific mood.

Referring to FIG. 5, a narrative in which a user does exercise (time-slot5), and after a meal (time-slot6), dances with friends (time-slot7) does not happen frequently socially (30%) or temporally and spatially (10%) during the entire data collection period.

However, it can be confirmed that these narratives are narratives that mainly have seasonal and temporal meanings appearing on the weekend in spring and make a positive mind state.

According to the embodiment of the present invention, the personal lifestyle narrative, the habit index derived from the lifestyle narrative, and the causal relationship analysis result between the user's mind state and the lifestyle may be used as a basis in presenting a method of taking appropriate measures necessary for a user's mental health management to a user, a user's guardian, a doctor, etc.

According to the embodiment of the present invention, by collecting objective data, the user's context is recognized, and the narrative of the user's personal lifestyle is derived.

This lifestyle narrative indicates a path of behavior and context to a specific mind state that a user wants to manage, and is generated based on more specific and numerous pieces of information than information used for managing an amount of exercise and a rest time.

A method of generating a narrative for lifestyle recognition according to the present invention includes (a) collecting and preprocessing user's biometric information, activity information, and environmental information, (b) forming an episode including contextual information including a user's behavior, social life, time and space, and mind state using the data preprocessed in operation (a), (c) grouping the episode to generate sequence data and interpreting the generated data as a user's lifestyle narrative, (d) extracting a habit pattern and a non-habit pattern using the narrative, and (e) analyzing a correlation between the user's lifestyle and mind state.

In operation (a), information including multi-modal sensor data and device usage log data is collected and preprocessed for synchronization based on time.

In operation (b), the episode is generated for each time period according to an order of generation of information and duration information.

In operation (c), the sequence data is generated by dividing the episode into groups of a preset time unit.

In operation (d), sequential pattern mining is applied to extract the habit pattern for a collected period, and a user's lifestyle narrative is generated as a pattern including consecutive events.

In operation (d), a habit index is extracted for each category of the contextual information.

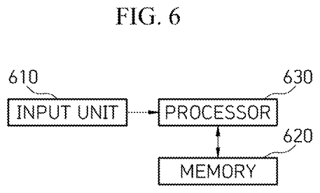

FIG. 6 illustrates an apparatus for generating a narrative for lifestyle recognition according to an embodiment of the present invention.

The apparatus for generating a narrative for lifestyle recognition according to the embodiment of the present invention includes an input unit 610 configured to collect basic data of narrative generation for lifestyle recognition, a memory 620 configured to store a program for generating a user's lifestyle narrative using the basic data, and a processor 630 configured to execute the program, in which the processor 630 performs a causal relationship analysis on a cause of a mind state and a result of the mind state using the lifestyle narrative.

The input unit 610 collects data including biometric information, environmental information, and activity information.

The processor 630 generates contextual information including a user's behavior, social life, time and space, and mind state and extracts a pattern for a habitual or non-habitual narrative.

The processor 630 extracts a habit pattern by applying sequential pattern mining and generates the user's lifestyle narrative using a pattern including consecutive events.

The processor 630 derives a habit index using the pattern and derives a lifestyle pattern, which affects a specific mind state, using the habit index.

The processor 630 calculates habit indexes for each of behavior, social relationship, time and space, and emotion.

According to another embodiment of the present invention, the processor 630 not only provides the causal relationship analysis result for the mind state using previously acquired data but also proposes a user's behavior pattern using the analysis result.

For example, in the above example (see FIG. 5), it is acquired as an analysis result that a narrative in which a user exercises and dances with friends after a meal is a narrative that makes the positive mind state is obtained as an analysis result.

When the information corresponding to the narrative has not been obtained within a recent period (for example, three months) and the user's mind state is continuously observed to be depressed, the processor 630 may present and recommend a behavior pattern corresponding to the corresponding narrative to the user in the current spring (for example, March to May) season in consideration of the analysis result.

For example, the processor 630 transmits a guide message to a user device to transmit a guide message about “It is the spring season when the weather is nice. How about having a round of golf with your acquaintances for the first time in a while and having a good time together at the club after a delicious meal?”

Through this, it is possible to provide a more active service by not only presenting the analysis result using the previously acquired data but also recommending a behavior pattern that can lead to a positive mind state to the user.

However, in providing these services, it is confirmed whether “the user can perform the corresponding behavior pattern” by using the previously acquired information.

For example, in a situation in which data indicating that the user is currently hospitalized for a long time due to a traffic accident is secured, transmitting the above-described guide message may have an adverse effect on the user.

Therefore, in this case, instead of providing a corresponding guide message, (when the user's prior approval is made), it is possible to provide pictures and videos of narratives that correspond to pleasant memories to the user's device along with the guide message “This is a photo with pleasant memories from last year at this time. Would you like to take a look for a change of mood?” like providing a timeline message using a pre-secured database.

Meanwhile, the method of generating a narrative for lifestyle recognition according to the embodiment of the present invention may be implemented in a computer system or recorded in a recording medium. The computer system may include at least one processor, a memory, a user input device, a data communication bus, a user output device, and storage. Each of the above-described components performs data communication through the data communication bus.

The computer system may further include a network interface coupled to a network. The processor may be a central processing unit (CPU) or a semiconductor device that processes instructions stored in the memory and/or storage.

The memory and storage may include various types of volatile or non-volatile storage media. For example, the memory may include a read only memory (ROM) and a random access memory (RAM).

Accordingly, the method of generating a narrative for lifestyle recognition according to the embodiment of the present invention may be implemented in a method executable by a computer. When the method of generating a narrative for lifestyle recognition according to the embodiment of the present invention is performed on a computer device, computer-readable instructions may perform the method of generating a narrative for lifestyle recognition according to the present invention.

Meanwhile, the method of generating a narrative for lifestyle recognition according to the present invention described above can be implemented as a computer-readable code on a computer-readable recording medium. The computer-readable recording medium includes any type of recording medium in which data readable by a computer system is stored. For example, there may be a ROM, a RAM, a magnetic tape, a magnetic disk, a flash memory, an optical data storage device, and the like. In addition, the computer-readable recording medium may be distributed in computer systems connected through a computer communication network, and stored and executed as readable codes in a distributed manner.

According to the present invention, by collecting objective data, it is possible to recognize a user's context and derive a narrative of a user's personal lifestyle.

By providing information less likely to be distorted or lost to a user himself/herself or a guardian, it is possible to use the information for mental health management of the user. By providing objective information to a professional counselor or doctor to support expert decision-making, it is possible to perform accurate diagnosis and appropriate treatment.

The effects of the present invention are not limited to the above-described effects, and other effects that are not mentioned may be obviously understood by those skilled in the art from the above detailed description.

The components described in the example embodiments may be implemented by hardware components including, for example, at least one digital signal processor (DSP), a processor, a controller, an application-specific integrated circuit (ASIC), a programmable logic element, such as an FPGA, other electronic devices, or combinations thereof. At least some of the functions or the processes described in the example embodiments may be implemented by software, and the software may be recorded on a recording medium. The components, the functions, and the processes described in the example embodiments may be implemented by a combination of hardware and software.

The method according to example embodiments may be embodied as a program that is executable by a computer, and may be implemented as various recording media such as a magnetic storage medium, an optical reading medium, and a digital storage medium.

Various techniques described herein may be implemented as digital electronic circuitry, or as computer hardware, firmware, software, or combinations thereof. The techniques may be implemented as a computer program product, i.e., a computer program tangibly embodied in an information carrier, e.g., in a machine-readable storage device (for example, a computer-readable medium) or in a propagated signal for processing by, or to control an operation of a data processing apparatus, e.g., a programmable processor, a computer, or multiple computers. A computer program(s) may be written in any form of a programming language, including compiled or interpreted languages and may be deployed in any form including a stand-alone program or a module, a component, a subroutine, or other units suitable for use in a computing environment. A computer program may be deployed to be executed on one computer or on multiple computers at one site or distributed across multiple sites and interconnected by a communication network.

Processors suitable for execution of a computer program include, by way of example, both general and special purpose microprocessors, and any one or more processors of any kind of digital computer. Generally, a processor will receive instructions and data from a read-only memory or a random access memory or both. Elements of a computer may include at least one processor to execute instructions and one or more memory devices to store instructions and data. Generally, a computer will also include or be coupled to receive data from, transfer data to, or perform both on one or more mass storage devices to store data, e.g., magnetic, magneto-optical disks, or optical disks. Examples of information carriers suitable for embodying computer program instructions and data include semiconductor memory devices, for example, magnetic media such as a hard disk, a floppy disk, and a magnetic tape, optical media such as a compact disk read only memory (CD-ROM), a digital video disk (DVD), etc. and magneto-optical media such as a floptical disk, and a read only memory (ROM), a random access memory (RAM), a flash memory, an erasable programmable ROM (EPROM), and an electrically erasable programmable ROM (EEPROM) and any other known computer readable medium. A processor and a memory may be supplemented by, or integrated into, a special purpose logic circuit.

The processor may run an operating system (OS) and one or more software applications that run on the OS. The processor device also may access, store, manipulate, process, and create data in response to execution of the software. For purpose of simplicity, the description of a processor device is used as singular; however, one skilled in the art will be appreciated that a processor device may include multiple processing elements and/or multiple types of processing elements. For example, a processor device may include multiple processors or a processor and a controller. In addition, different processing configurations are possible, such as parallel processors.

Also, non-transitory computer-readable media may be any available media that may be accessed by a computer, and may include both computer storage media and transmission media.

The present specification includes details of a number of specific implements, but it should be understood that the details do not limit any invention or what is claimable in the specification but rather describe features of the specific example embodiment. Features described in the specification in the context of individual example embodiments may be implemented as a combination in a single example embodiment. In contrast, various features described in the specification in the context of a single example embodiment may be implemented in multiple example embodiments individually or in an appropriate sub-combination. Furthermore, the features may operate in a specific combination and may be initially described as claimed in the combination, but one or more features may be excluded from the claimed combination in some cases, and the claimed combination may be changed into a sub-combination or a modification of a sub-combination.

Similarly, even though operations are described in a specific order on the drawings, it should not be understood as the operations needing to be performed in the specific order or in sequence to obtain desired results or as all the operations needing to be performed. In a specific case, multitasking and parallel processing may be advantageous. In addition, it should not be understood as requiring a separation of various apparatus components in the above described example embodiments in all example embodiments, and it should be understood that the above-described program components and apparatuses may be incorporated into a single software product or may be packaged in multiple software products.

It should be understood that the example embodiments disclosed herein are merely illustrative and are not intended to limit the scope of the invention. It will be apparent to one of ordinary skill in the art that various modifications of the example embodiments may be made without departing from the spirit and scope of the claims and their equivalents.

What is claimed is:

1. A system for generating a narrative for lifestyle recognition, comprising:
a server configured to receive and store multi-modal sensor data and user's device usage log data;
a computing device configured to call the multi-modal sensor data and the device usage log data from the server to recognize contextual information, generate a user's personal narrative, and derive lifestyle information; and
a display device configured to perform visualization using the lifestyle information and provide an option to confirm an analysis result.

2. The system of claim 1, wherein the multi-modal sensor data includes biometric information and environmental information.

3. The system of claim 1, wherein the computing device preprocesses the multi-modal sensor data and the device usage log data to be synchronized and integrated based on time.

4. The system of claim 1, wherein the computing device recognizes the contextual information, which includes a user's behavior, social life, time and space, and mind state, by time.

5. The system of claim 4, wherein the computing device classifies the narrative based on a predefined time and extracts patterns for a habit narrative and a non-habit narrative.

6. The system of claim 5, wherein the computing device derives a habit index using the patterns for the habit narrative and the non-habit narrative and derives a lifestyle pattern, which affects the mind state, using the habit index.

7. The system of claim 1, wherein the display device provides a responsive user interface to which at least one option of an inquiry period, a time interval, confirmation of analysis results for each mind state, and confirmation of analysis results by location is applied.

8. A method of generating a narrative for lifestyle recognition, comprising:
(a) collecting and preprocessing user's biometric information, activity information, and environmental information;
(b) forming an episode including contextual information including a user's behavior, social life, time and space, and mind state using data preprocessed in operation (a);
(c) grouping the episode to generate sequence data and interpreting the generated data as a user's lifestyle narrative;
(d) extracting a habit pattern and a non-habit pattern using the narrative; and
(e) analyzing a correlation between the user's lifestyle and mind state.

9. The method of claim 8, wherein, in operation (a), information including multi-modal sensor data and device usage log data is collected and preprocessed for synchronization based on time.

10. The method of claim 8, wherein, in operation (b), the episode is generated for each time period according to an order of generation of information and duration information.

11. The method of claim 8, wherein, in operation (c), the sequence data is generated by dividing the episode into groups of a preset time unit.

12. The method of claim 8, wherein, in operation (d), sequential pattern mining is applied to extract the habit pattern for a collected period, and a user's lifestyle narrative is generated as a pattern including consecutive events.

13. The method of claim 8, wherein, in operation (d), a habit index is extracted for each category of the contextual information.

14. An apparatus for generating a narrative for lifestyle recognition, comprising:
an input unit configured to collect basic data of narrative generation for the lifestyle recognition;
a memory configured to store a program for generating a user's lifestyle narrative using the basic data; and
a processor configured to execute the program,
wherein the processor performs a causal relationship analysis on a cause of a mind state and a result of the mind state using the lifestyle narrative.

15. The apparatus of claim 14, wherein the input unit collects data including biometric information, environmental information, and activity information.

16. The apparatus of claim 14, wherein the processor generates contextual information including a user's behavior, social life, time and space, and mind state, and extracts a pattern for a habit or non-habit narrative.

17. The apparatus of claim 16, wherein the processor extracts a habit pattern by applying sequential pattern mining and generates the user's lifestyle narrative using a pattern including consecutive events.

18. The apparatus of claim 17, wherein the processor derives a habit index using the pattern and derives a lifestyle pattern, which affects a specific mind state, using the habit index.

19. The apparatus of claim 18, wherein the processor calculates the habit indexes for each of behavior, social relationship, time and space, and emotion.
